(19) United States
(12) Patent Application Publication
(10) Pub. No.: US 2025/0205504 A1
(43) Pub. Date: Jun. 26, 2025

(54) DEFIBRILLATOR AND DEFIBRILLATION ENERGY CHARGING METHOD

(71) Applicant: FUKUDA DENSHI CO., LTD., Tokyo (JP)

(72) Inventors: Ryosuke SAKAI, Tokyo (JP); Katsuya IDE, Tokyo (JP); Taichi IWASAKI, Tokyo (JP); Koki OYA, Tokyo (JP); Takashi YAMAKI, Tokyo (JP); Kohei MITSUMARU, Tokyo (JP); Hiroshi HINO, Tokyo (JP); Yuzuru SAKAI, Tokyo (JP)

(21) Appl. No.: 18/989,045

(22) Filed: Dec. 20, 2024

(30) Foreign Application Priority Data
Dec. 26, 2023 (JP) 2023-219909

(51) Int. Cl. A61N 1/39; A61N 1/362

(52) U.S. Cl. CPC A61N 1/3975; A61N 1/3625; A61N 1/3904

(57) ABSTRACT

A defibrillator of the present disclosure includes: a first battery; a second battery; a capacitor that stores defibrillation energy; a voltage analyzer that measures a voltage of the first battery during charging of the capacitor by using the first battery; and a controller that controls, based on the voltage measured by the voltage analyzer, whether to use the second battery for charging the capacitor.

### CROSS REFERENCE TO RELATED APPLICATIONS

This application is entitled to and claims the benefit of Japanese Patent Application No. 2023-219909, filed on Dec. 26, 2023, the disclosure of which including the specification, drawings and abstract is incorporated herein by reference in its entirety.

### TECHNICAL FIELD

The present disclosure relates to a defibrillator and a defibrillation energy charging method, in particular, relates to a defibrillator that charges a capacitor with defibrillation energy by using a battery.

### BACKGROUND ART

A defibrillator has been widely used as an apparatus for treating ventricular fibrillation (Vf). An automated external defibrillator (AED) is well known as a representative defibrillator. The defibrillator treats ventricular fibrillation or pulseless ventricular tachycardia (pulseless VT) of a patient who suffers from the ventricular fibrillation or pulseless ventricular tachycardia, by discharging a pulse of a high voltage into the heart of the patient.

Such a defibrillator includes a battery, a capacitor, and a power switch. Before the defibrillation is performed, the capacitor is charged by the battery. The capacitor is electrically connected to a discharger such as an electrode pad or a paddle via the power switch. When the power switch is turned on, the defibrillation energy stored in the capacitor is discharged through the discharger to the patient's heart.

Such a defibrillator is described in, for example, Japanese Patent Application Laid-Open No. 2016-187438.

When the battery is depleted (in other words, when the remaining battery level drops), there is a challenge in that the time until the capacitor is charged with the desired energy becomes long. Second, there is a possibility that the capacitor may not be charged with a sufficient amount of energy.

As a result, when the battery is depleted, there is a possibility that defibrillation cannot be performed at an appropriate timing and with an appropriate amount of energy. In particular, it is necessary to promptly treat patients suffering from ventricular fibrillation and pulseless ventricular tachycardia, and thus, it is necessary to charge energy to the capacitor at a high speed. Accordingly, it is preferable to solve the topic described above.

The present disclosure has been made in view of the above points, and an object thereof is to provide a defibrillator and a defibrillation energy charging method each capable of performing defibrillation at an appropriate timing and with an appropriate amount of energy.

### SUMMARY

An aspect of a defibrillator of the present disclosure includes:
a first battery;
a second battery;
a capacitor that stores defibrillation energy;
a voltage analyzer that measures a voltage of the first battery during charging of the capacitor by using the first battery; and
a controller that controls, based on the voltage measured by the voltage analyzer, whether to use the second battery for the charging of the capacitor.

An aspect of a defibrillation energy charging method of the present disclosure is a defibrillation energy charging method in a defibrillator, and includes:
charging a capacitor by a first battery;
measuring an output voltage of the first battery during the charging of the capacitor by using the first battery; and
controlling, based on the measured output voltage of the first battery, whether to charge the capacitor by using a second battery.

### BRIEF DESCRIPTION OF THE DRAWINGS

FIG. 1 is a block diagram illustrating a main configuration of a defibrillator in an embodiment;

FIG. 2 is a flowchart for describing a defibrillation energy charging operation in the embodiment;

FIGS. 3A to 3E are diagrams illustrating changes in voltage, current, and defibrillation energy during charging of the defibrillation energy in the embodiment, in which FIG. 3A illustrates the output voltage of a main battery, FIG. 3B illustrates the output current of the main battery, FIG. 3C illustrates the output voltage of a sub-battery, FIG. 3D illustrates the output current of the sub-battery, and FIG. 3E illustrates the voltage between both ends of a capacitor;

FIGS. 4A to 4C are diagrams illustrating the output voltage of the main battery (FIG. 4A), the output current of the main battery (FIG. 4B), and the voltage between both ends of the capacitor (FIG. 4C) in a case where the remaining battery level of the main battery is sufficient;

FIGS. 5A to 5C are diagrams illustrating an example in which the time required for charging the defibrillation energy becomes long, in which FIG. 5A illustrates the output voltage of the main battery, FIG. 5B illustrates the output current of the main battery, and FIG. 5C illustrates the voltage between both ends of the capacitor; and

FIGS. 6A to 6C are diagrams illustrating an example in which a desired amount of defibrillation energy cannot be charged due to a momentary interruption, in which FIG. 6A illustrates the output voltage of the main battery, FIG. 6B illustrates the output current of the main battery, and FIG. 6C illustrates the voltage between both ends of the capacitor.

### DESCRIPTION OF EMBODIMENTS

Hereinafter, an embodiment of the present disclosure will be described in detail with reference to the drawings.

### <1> Main Configuration of Defibrillator

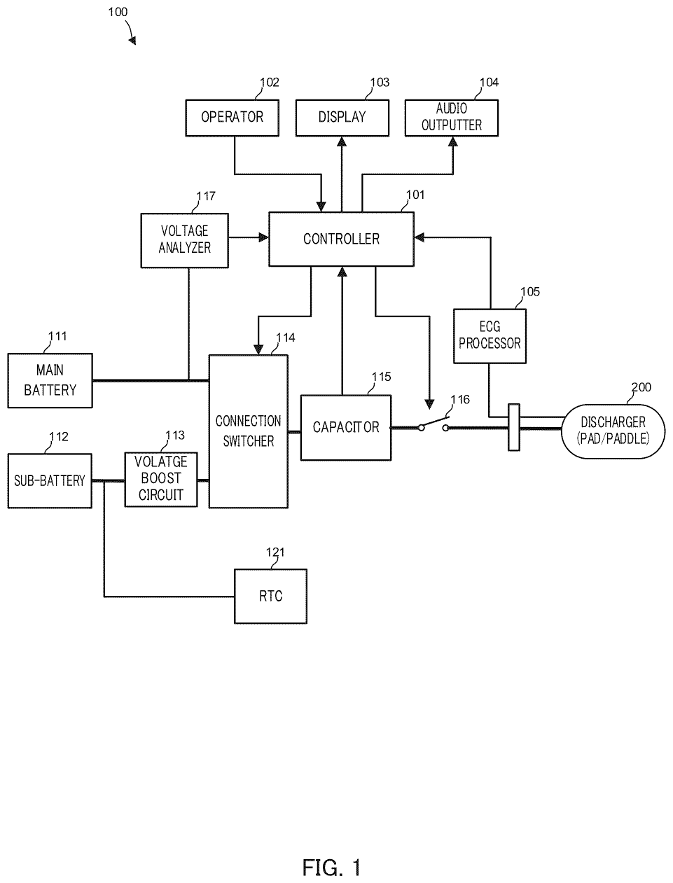

FIG. 1 is a block diagram illustrating a main configuration of defibrillator 100 in the present embodiment. Defibrillator 100 is what is called an external defibrillator or an AED. Discharger 200 is connected to defibrillator 100. Discharger 200 is an electrode pad or a paddle that comes into contact with a patient.

Defibrillator 100 includes controller 101, operator 102, display 103, audio outputter 104, and electrocardiogram (ECG) processor 105. In addition, defibrillator 100 includes main battery 111, sub-battery 112, voltage boost circuit 113, connection switcher 114, capacitor 115, power switch 116, and voltage analyzer 117.

Controller 101 is implemented by a processor such as a central processing unit (CPU), and controls the operation of each section of defibrillator 100. Further, controller 101 implemented by the processor realizes, for example, a defibrillation energy charging method to be described later by executing a program.

Operator 102 includes, for example, a charge button, a shock button, and the like (not illustrated). Note that, in a case where discharger 200 is a paddle type, the shock button may be provided on the paddle. When the charge button is operated, charging of capacitor 115 is started, and when the shock button is operated, the electric charge stored in capacitor 115 is output to discharger 200.

Further, defibrillator 100 is configured such that controller 101 can perform electrocardiogram analysis and also determine the necessity of defibrillation or the like based on the electrocardiogram acquired by ECG processor 105.

Main battery 111 is provided for charging capacitor 115 and generating defibrillation energy. Sub-battery 112 is mainly used as a power supply other than defibrillation energy such as real time clock (RTC) 121.

In the example of the present embodiment, both main battery 111 and sub-battery 112 are lithium primary batteries. Further, in the example of the present embodiment, the battery capacities of main battery 111 and sub-battery 112 are equal. The output voltage of main battery 111 is, however, greater than the output voltage of sub-battery 112.

Main battery 111 is a battery replaceably mounted in defibrillator 100 (that is, a replaceable battery), and is replaced by a new battery that has been fully charged, for example, every two years. Sub-battery 112 is, on the other hand, a battery attached to defibrillator 100 in a non-replaceable manner (that is, a built-in battery). Main battery 111 has a battery capacity capable of, for example, charging capacitor 115 approximately 200 times. Sub-battery 112 has a battery capacity capable of, for example, operating the internal circuit of defibrillator 100 such as RTC 121 for 10 years.

Main battery 111 used as described above has an output voltage of 12 to 18 [V] in a state where no load (capacitor 115) is connected. In the example of the present embodiment, the output voltage of main battery 111 is 15 [V] in a state where no load is connected. Sub-battery 112 used as described above has an output voltage of 2 to 5 [V] in a state where no load is connected. In the example of the present embodiment, the output voltage of sub-battery 112 is 3 [V] in a state where no load is connected. Voltage boost circuit 113 boosts the 3 [V] voltage input from sub-battery 112 to approximately 15 [V] and outputs the voltage.

Voltage analyzer 117 measures the output voltage of main battery 111 when capacitor 115 is being charged by main battery 111. Information on the voltage measured by voltage analyzer 117 is output to controller 101. Note that, various voltage measurement circuits proposed heretofore can be applied as the configuration of voltage analyzer 117. For example, voltage analyzer 117 can be realized by a voltage measurement circuit including a monitoring IC (Integrated Circuit).

Controller 101 controls, based on the voltage measured by voltage analyzer 117, which of main battery 111 and sub-battery 112 is used for charging capacitor 115. Specifically, controller 101 controls whether to connect only main battery 111 to capacitor 115 or to connect both main battery 111 and sub-battery 112 to capacitor 115, by outputting a control signal to connection switcher 114. Connection switcher 114 is configured with, for example, a power path control IC, a voltage boost circuit that boosts a battery voltage, an energy output module, and the like.

In the case of the present embodiment, controller 101 determines whether the output voltage of main battery 111, as measured by voltage analyzer 117, is equal to or greater than a predetermined threshold. Then, in a case where the output voltage of main battery 111 is equal to or greater than the threshold, controller 101 causes capacitor 115 and main battery 111 to be connected to each other, and causes capacitor 115 to be charged by using main battery 111. In contrast, in a case where the output voltage of main battery 111 is less than the threshold, controller 101 causes both main battery 111 and sub-battery 112 to be connected to capacitor 115, and causes capacitor 115 to be charged by using not only main battery 111 but also sub-battery 112.

Further, controller 101 calculates the defibrillation energy stored in capacitor 115 based on the voltage between both ends of the high-voltage capacitor that constitutes capacitor 115. When the defibrillation energy is equal to or greater than a predetermined value and the shock button is operated, controller 101 turns on power switch 116 and causes the electric charge stored in capacitor 115 to be output to discharger 200.

### <2> Operation of Embodiment

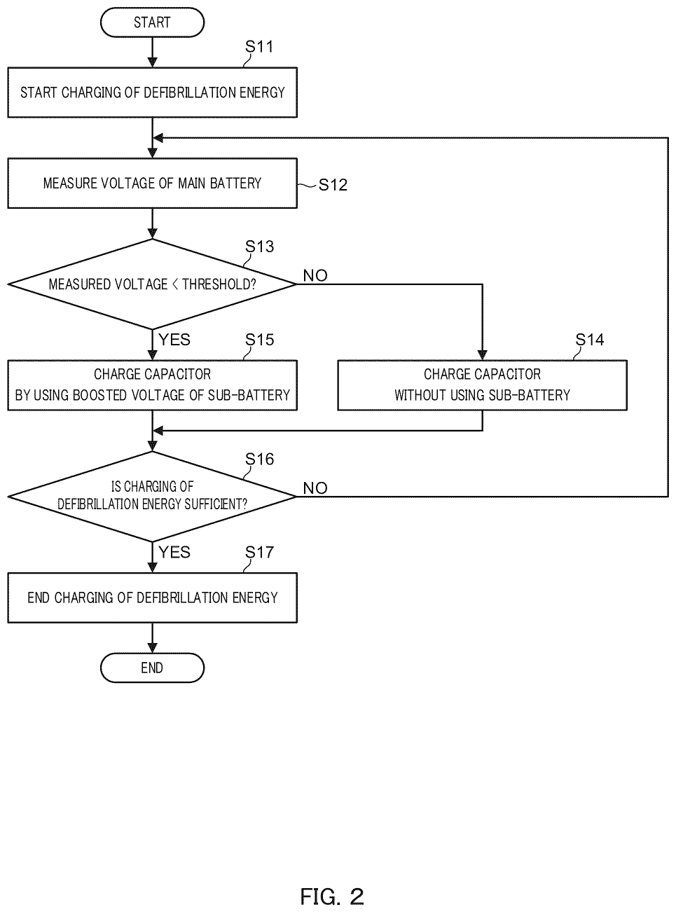

FIG. 2 is a flowchart for describing a defibrillation energy charging operation in defibrillator 100.

When the charge button is operated by the user, defibrillator 100 starts charging of the defibrillation energy in step S11. At this time, when controller 101 controls connection switcher 114 such that main battery 111 and capacitor 115 are connected to each other, capacitor 115 is charged by main battery 111.

In subsequent step S12, the voltage of main battery 111 is measured by voltage analyzer 117. In step S13 that follows, controller 101 compares the measured voltage with a threshold and determines whether the measured voltage is equal to or greater than the threshold.

In a case where the measured voltage is equal to or greater than the threshold (step S13; NO), defibrillator 100 proceeds to step S14 and charges capacitor 115 without using sub-battery 112. In other words, capacitor 115 is charged by using only main battery 111.

In a case where the measured voltage is less than the threshold (step S13; YES), on the other hand, defibrillator 100 proceeds to step S15, and charges capacitor 115 by using the voltage of sub-battery 112 boosted by voltage boost circuit 113. At this time, main battery 111 may also be used together with sub-battery 112 for charging, or only sub-battery 112 may be used for charging. In the case of the present embodiment, main battery 111 is also used together to charge capacitor 115.

In step S16 that follows, controller 101 determines whether the charging of the defibrillation energy is sufficient, based on the voltage between both ends of capacitor 115. When it is determined that a sufficient amount of defibrillation energy is charged into capacitor 115 (step S16; YES), defibrillator 100 proceeds to step S17 and ends the charging of the defibrillation energy into capacitor 115. In contrast, when it is determined that capacitor 115 is not charged with a sufficient amount of defibrillation energy (step S16; NO), defibrillator 100 returns to step S12 and continues the charging.

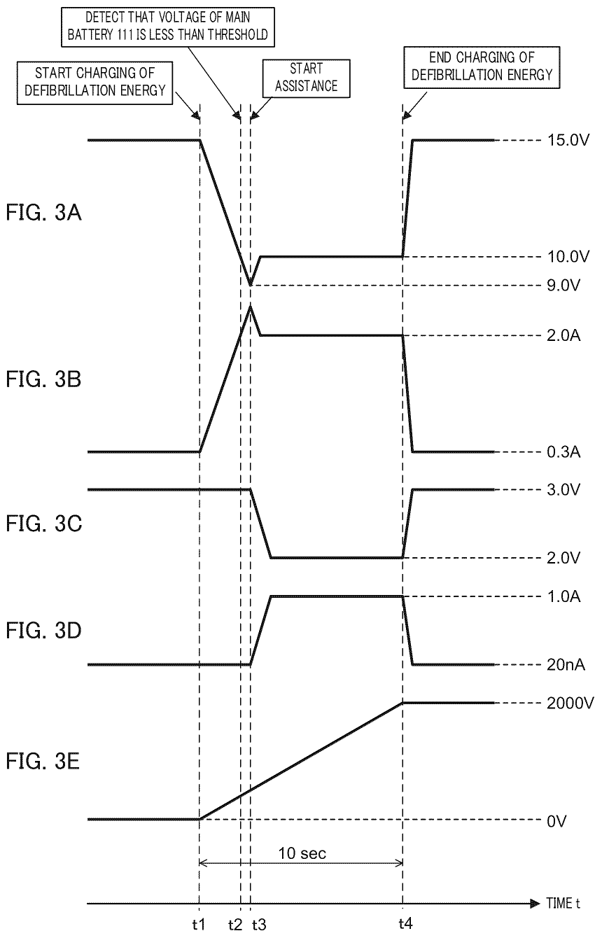

FIGS. 3A to 3E are diagrams illustrating changes in voltage, current, and defibrillation energy during charging of defibrillation energy by defibrillator 100 in the present embodiment.

FIG. 3A illustrates the output voltage of main battery 111, FIG. 3B illustrates the output current of main battery 111, FIG. 3C illustrates output voltage of sub-battery 112, FIG. 3D illustrates the output current of sub-battery 112, and FIG. 3E illustrates the voltage between both ends of capacitor 115 (that is, the voltage corresponding to the defibrillation energy stored in capacitor 115).

Defibrillator 100 starts charging the defibrillation energy by connecting main battery 111 to capacitor 115 at time t1. In this case, the output voltage of main battery 111 drops rapidly.

Here, since primary batteries have high output impedance, using a primary battery as main battery 111, as in the embodiment, and connecting a large load, such as capacitor 115 of defibrillator 100, to the output side of the primary battery cause a rapid voltage drop. As described above, this voltage drop becomes larger as main battery 111 is more depleted.

When defibrillator 100 detects that the voltage of main battery 111 is less than the threshold at time t2, defibrillator 100 starts assistance using sub-battery 112 at a subsequent time t3. Note that, in the example in FIGS. 3A to 3E, the threshold is 10 [V].

Defibrillator 100 charges capacitor 115 by applying not only the voltage of main battery 111 but also the boosted voltage of sub-battery 112 to capacitor 115 during the period from time t3 to time t4. In the example of the present embodiment, voltage boost circuit 113 boosts the output voltage of sub-battery 112 to 10 [V] and applies the 10 [V] output voltage to capacitor 115. In reality, the output voltages of main battery 111 and sub-battery 112 are further boosted by a voltage boost circuit (not illustrated) in connection switcher 114 and then applied to capacitor 115.

In this manner, even in a case where main battery 111 is depleted (that is, the remaining battery level is insufficient), and the output voltage significantly drops during the charging of capacitor 115, defibrillator 100 of the present embodiment can avoid situations where the time required for charging the defibrillation energy becomes long or where the necessary amount of the defibrillation energy cannot be charged, by assisting main battery 111 by using sub-battery 112.

### <3> Comparative Example

Here, an operation of a defibrillator in which the defibrillation energy is charged by using only main battery 111, without the assistance in charging the defibrillation energy by using sub-battery 112, will be described as a comparative example with respect to the embodiment.

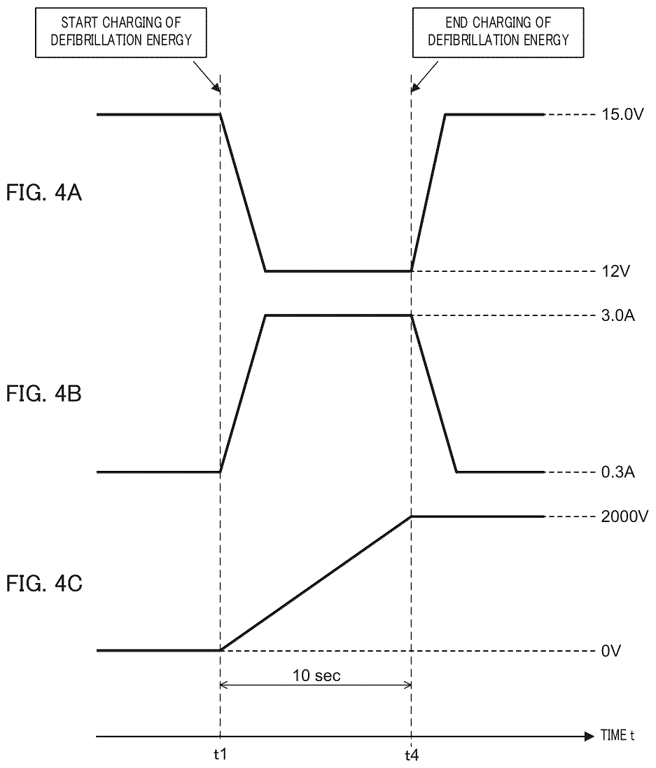

FIGS. 4A to 4C are an example in a case where the remaining battery level of main battery 111 is sufficient. FIG. 4A is a diagram illustrating the output voltage of main battery 111, FIG. 4B is a diagram illustrating the output current of main battery 111, and FIG. 4C is a diagram illustrating the voltage between both ends of capacitor 115 (that is, the voltage corresponding to the defibrillation energy stored in capacitor 115).

When main battery 111 is connected to capacitor 115 at time t1 and the charging of the defibrillation energy is started, the output voltage of main battery 111 drops, but the drop is small since the remaining battery level is sufficient. In the example of FIG. 4A, the voltage of main battery 111 during charging is 12 [V], which is a voltage capable of charging capacitor 115 within a predetermined time. Thus, the time required for charging the defibrillation energy (time from the start of charging to the end of charging) is a designated time (10 [sec]), and does not become long.

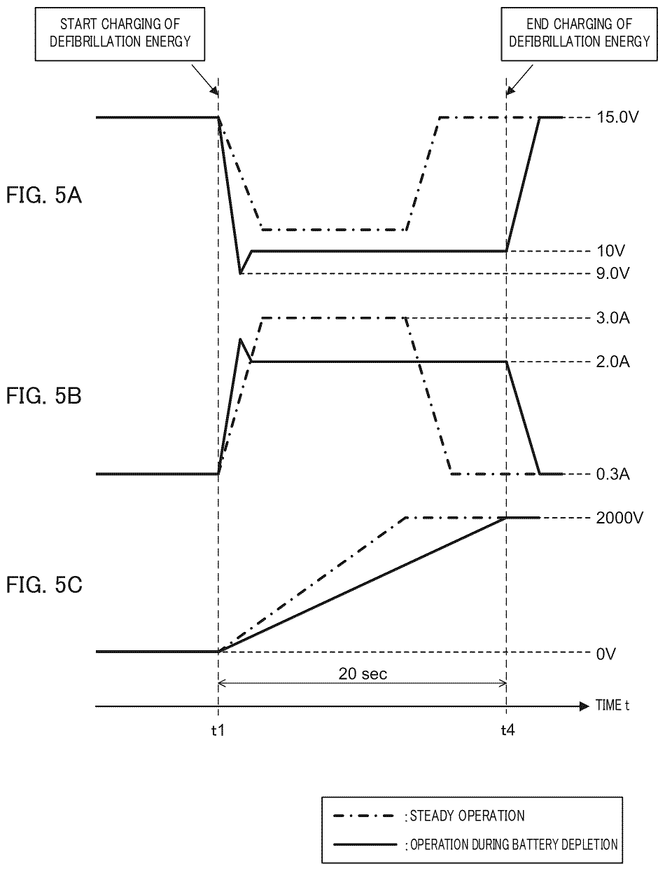

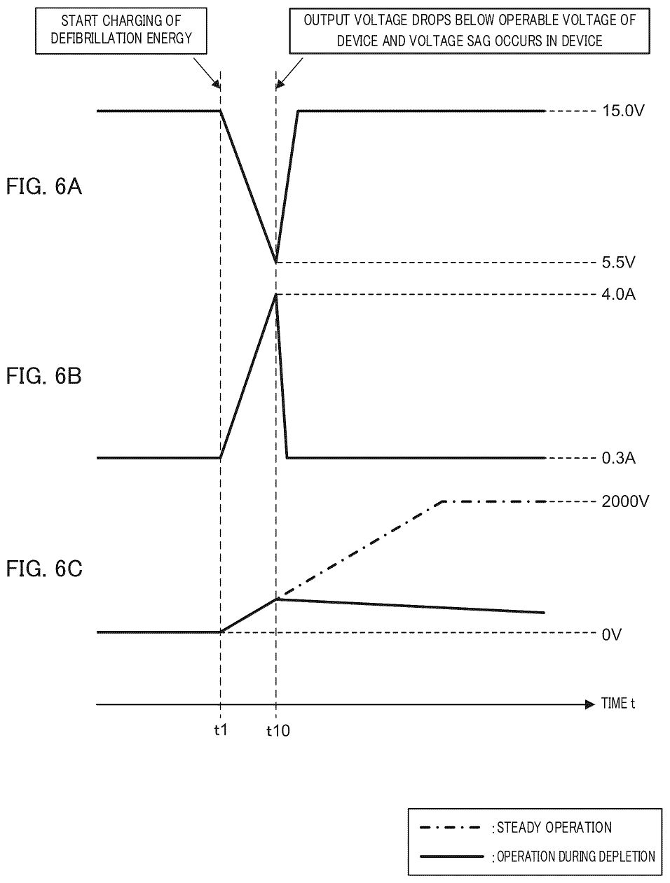

In a case where the remaining battery level of main battery 111 is not sufficient, on the other hand, a challenge occurs. FIGS. 5A to 5C illustrate an example in which the time required for charging the defibrillation energy becomes long, and FIGS. 6A to 6C illustrate an example in which a desired amount of defibrillation energy cannot be charged.

First, the example of FIGS. 5A to 5C will be described. In FIGS. 5A to 5C, the solid line indicates an operation during battery depletion, and the dash-dotted line indicates an operation in a steady state (that is, a state when the battery is not depleted) for comparison. FIG. 5A is a diagram illustrating the output voltage of main battery 111, FIG. 5B is a diagram illustrating the output current of main battery 111, and FIG. 5C is a diagram illustrating the voltage between both ends of capacitor 115 (that is, the voltage corresponding to the defibrillation energy stored in capacitor 115).

When the charging of the defibrillation energy is started by connecting main battery 111 to capacitor 115 at time t1, the output voltage of main battery 111 drops rapidly. In particular, in the example of FIGS. 5A to 5C, main battery 111 is being depleted, and thus, the output voltage drops significantly. If the output voltage of main battery 111 drops too much, other parts (for example, the CPU) to which voltage is applied by main battery 111 will stop operating. Therefore, a process of limiting the output current of main battery 111 is performed to prevent the operations of the other parts from stopping. As illustrated in FIG. 5B, the output current is limited from 3 [A] to 2 [A] in this example. As a result, the rate of electric charge accumulation in capacitor 115 decreases, and as illustrated in FIG. 5C, the time required for charging the defibrillation energy becomes long. In the embodiment, the charging time of the defibrillation energy is 10 [sec], but the charging time in the present example is 20 [sec].

Next, the example in FIGS. 6A to 6C will be described. In FIGS. 6A to 6C, the solid line indicates an operation during battery depletion, and the dash-dotted line indicates an operation in a steady state (that is, a state when the battery is not depleted) for comparison. FIG. 6A is a diagram illustrating the output voltage of main battery 111, FIG. 6B is a diagram illustrating the output current of main battery 111, and FIG. 6C is a diagram illustrating the voltage between both ends of capacitor 115 (that is, the voltage corresponding to the defibrillation energy stored in capacitor 115).

When the charging of the defibrillation energy is started by connecting main battery 111 to capacitor 115 at time t1, the output voltage of main battery 111 drops rapidly. In particular, in the example of FIGS. 6A to 6C, main battery 111 is being depleted, and thus, the output voltage drops significantly. As described in FIGS. 5A to 5C, the drop in the output voltage is prevented when the current is limited, but the output voltage significantly drops in a case where the current is not limited. In the example of FIG. 6A, the output voltage drops to 5.5 [V] at time t10. As a result, other parts (for example, the CPU) to which voltage is applied by main battery 111 will stop operating (so-called “voltage sag” occurs), and the defibrillator will stop operating. Thus, as illustrated in FIG. 6C, a desired amount of defibrillation energy cannot be charged into the capacitor.

As described above, defibrillator 100 of the present embodiment includes: a first battery (main battery 111); a second battery (sub-battery 112); capacitor 115 that stores defibrillation energy; voltage analyzer 117 that measures a voltage of the first battery during charging of capacitor 115 by using the first battery; and controller 101 that controls, based on the voltage measured by voltage analyzer 117, whether to use the second battery for charging capacitor 115.

Thus, in a case where the first battery is depleted, capacitor 115 is charged by using the second battery, and thus, it is possible to avoid that the time for charging the defibrillation energy becomes long and that a sufficient amount of defibrillation energy cannot be charged, even in a case where the first battery is depleted. Thus, defibrillator 100 capable of performing defibrillation at an appropriate timing and with an appropriate amount of energy can be realized.

Further, capacitor 115 is charged by using the second battery only when the first battery is depleted, and thus, it is possible to prevent the depletion of the second battery. Accordingly, in a case where, as in the embodiment, the second battery (sub-battery 112) is a built-in (that is, non-replaceable) battery, it is possible to prevent the second battery (sub-battery 112) from being unnecessarily depleted, and it is possible to prevent the product life of defibrillator 100 from being shortened.

In addition, defibrillator 100 may be placed not only indoors but also outdoors, and in such cases, when the temperature is low, a voltage drop of main battery 111 becomes remarkable. Accordingly, the charging of the defibrillation energy is significantly affected not only by the depletion of main battery 111 but also by the external environment.

Even in such cases, defibrillator 100 of the present embodiment boosts the output voltage of sub-battery 112 and applies the boosted output voltage of sub-battery 112 to capacitor 115 to charge capacitor 115 with the defibrillation energy in a case where the output voltage of main battery 111 becomes less than a predetermined value. Accordingly, it is possible to avoid that the time for charging the defibrillation energy becomes long and that a sufficient amount of defibrillation energy cannot be charged.

The embodiment described above is only illustration of an exemplary embodiment for implementing the present invention, and the technical scope of the present invention shall not to be construed limitedly thereby. That is, the present invention can be implemented in various forms without departing from the gist or the main features thereof.

In the embodiment described above, a case where it is determined whether the output voltage of main battery 111 is equal to or greater than a predetermined threshold has been described, but the present disclosure is not limited thereto. The inclination of the output voltage of main battery 111 may be measured, and in a case where the inclination becomes steeper than a predetermined value, it may be determined that main battery 111 is being depleted, and the sub-battery 112 may be used. In this manner, it is possible to start charging using sub-battery 112 before the output voltage of main battery 111 drops below a predetermined value, and thus, it is possible to further shorten the charging time.

According to the present disclosure, it is possible to provide a defibrillator and a defibrillation energy charging method capable of performing defibrillation at an appropriate timing and with an appropriate energy.

### REFERENCE SIGNS LIST

100 Defibrillator
101 Controller
111 Main battery
112 Sub-battery
113 Voltage boost circuit
114 Connection switcher
115 Capacitor
116 Power switch
117 Voltage analyzer
200 Discharger (pad or paddle)

1. A defibrillator, comprising:
a first battery;
a second battery;
a capacitor that stores defibrillation energy;
a voltage analyzer that measures a voltage of the first battery during charging of the capacitor by using the first battery; and
a controller that controls, based on the voltage measured by the voltage analyzer, whether to use the second battery for the charging of the capacitor.

2. The defibrillator according to claim 1, further comprising a voltage boost circuit provided between the second battery and the capacitor, wherein:
a voltage of the second battery is smaller than the voltage of the first battery, and
the voltage of the second battery is applied to the capacitor via the voltage boost circuit.

3. The defibrillator according to claim 1, wherein
the controller is configured to determine whether the measured voltage of the first battery is equal to or greater than a predetermined threshold and control the charging of the capacitor such that the capacitor is charged by using the second battery in a case where the voltage of the first battery is less than the threshold.

4. The defibrillator according to claim 1, wherein
the controller is configured to control the charging of the capacitor such that the capacitor is charged by using the second battery in a case where steepness of a voltage drop in the measured voltage of the first battery is greater than a predetermined threshold.

5. The defibrillator according to claim 1, wherein
the first battery and the second battery are primary batteries.

6. The defibrillator according to claim 1, wherein
the first battery is a replaceable battery, and the second battery is a built-in battery.

7. A defibrillation energy charging method in a defibrillator, the method comprising:
charging a capacitor by a first battery;
measuring an output voltage of the first battery during the charging of the capacitor by using the first battery; and
controlling, based on the measured output voltage of the first battery, whether to charge the capacitor by using a second battery.
